(19) United States
(12) Patent Application Publication
(10) Pub. No.: US 2015/0280977 A1
(43) Pub. Date: Oct. 1, 2015

(54) METHOD AND A SYSTEM FOR ATTACHING A WIRELESS OR NON-WIRELESS DEVICE TO A PLATFORM AND ESTABLISHING COMMUNICATION THERE WITH

(71) Applicant: TELEFONICA, S.A., Madrid (ES)

(72) Inventors: Luis Curiel, Madrid (ES); Helio Huete, Madrid (ES)

(73) Assignee: TELEFONICA, S.A., Madrid (ES)

(21) Appl. No.: 14/431,104

(22) PCT Filed: Sep. 18, 2013
(86) PCT No.: PCT/EP2013/069342

§ 371 Date: Mar. 25, 2015

(30) Foreign Application Priority Data
Sep. 27, 2012 (ES) P201231493

(51) Int. Cl. H04L 12/24; H04L 29/06; H04L 29/08

(52) U.S. Cl. CPC H04L 41/0803; H04L 67/12; H04L 65/1013; H04W 84/18

(57) ABSTRACT

The method comprises providing a gateway with configuration information regarding an unknown device to be attached thereto, the method comprising:
providing a Dynamic Pair-up function in said gateway;
using, the gateway, said Dynamic Pair-up function to retrieve said configuration information from a data source; and
using, the gateway, said retrieved configuration information to automatically establish communication with said device.

The system is configured for implementing the method of the invention.

### FIELD OF THE ART

This invention generally relates, in first aspect, to a method for attaching a wireless or non-wireless device to a platform and establishing communication there with, comprising providing a gateway with configuration information regarding a wireless or non-wireless device to be attached thereto, and more particularly to a method applied to the attaching of a wireless or non-wireless device which is unknown for the gateway, which avoids the need of providing the device also with configuration information, therefore only needing the providing of configuration information to the gateway.

A second aspect of the invention concerns to a system configured for implementing the method of the first aspect.

### BACKGROUND OF THE PRESENT INVENTION

The world is getting full of sensors [1]. They are everywhere colonizing people's life at home, in the cars, at the hospitals, etc. since they have countless areas of usage. As their number increases every year, the interoperability problem raises as one of their main hurdles for service providers. Adding new sensors to a platform is a non-solved problem, which makes products cumbersome providing tricky solutions for end-users.

If we look more specifically into the e-Health device market, interoperability is precisely one of the biggest problems. After some years, manufacturer companies' adoption of Continua Alliance standards is still terribly low. This is partly because these companies have to cope with the specific implementations of adding the Continua profiles in the devices' protocol stacks (e.g. Continua Bluetooth profile) in both sensors and gateways, which makes the whole process very tedious and expensive and in turn, devices end up being also more expensive. As well as that, Continua is specialized only in the e-Health market which gives it a very limited vision of the sensors' integration problem.

Furthermore, Bluetooth low energy [2] has got into the arena detracting the value of the existing standards and increasing the feeling of failure of the current standards. For this and all the previous reasons, device companies carry on producing new sensors and gateways following their proprietary solutions. In the meantime, Platform Providers struggle every day to attach new devices to their systems, spending a lot of time and money in order to have the best and most complete solution. Each system provides its own solution and for platform designers it's a nightmare to integrate different devices.

Therefore, speeding up time to market and increase the flexibility to adapt new devices is still a key point for platforms to be successful. Even though Continua intends to solve this problem, manufacturers are not adopting the standards as fast as it was expected, so a flexible and fast need to integrate difference sorts of devices is needed.

Next some prior art documents which disclose different methods and systems for attaching wireless devices to a platform via a gateway are cited and briefly described.

US20100016683 discloses a system for installing a remote healthcare system including a set top box (STB) and a home gateway configured by inserting a configuration carrier into each device. The configuration carrier has stored thereon configuration routines and information provided by the remote health monitoring service provider. Configuration information includes, without being limited to, network IDs and channel information, hardware ID information, and the like. The home gateway is connected to the STB using a network link and to a central server over an Ethernet link. The home gateway is further coupled to one or more patient monitoring devices using a wireless link, such as Bluetooth.

US20100016683 describes a method to set up a monitoring system and is not focused on the acquisition of data that subsequently can be processed and use it in a platform of any kind. Moreover, the system described in said application knows beforehand which biometric devices can communicate with and how to perform this communication.

EP1850527A1 describes an invention related to the enabling/configuring of a service on a residential gateway, whereby the residential gateway receives a signal from a data carrier, which signal triggers the residential gateway to enable the service and start configuring it. The residential gateway receives information-data from the data carrier, which information-data comprises information about the service to be configured. A configuration-request-message is sent from the residential gateway to a configuration server of a service provider, which configuration-request-message comprises information about the service to be configured. Configuration-data is received and stored in the residential gateway from the configuration server, thereby enabling usage of the service on the residential gateway.

The main focus of EP1850527A1 system is describing a way to provide and set up services that knows beforehand. Furthermore, in the system of EP1850527A1 services must be enabled and provided with some configuration information in order for the gateway to communicate with them.

### DESCRIPTION OF THE INVENTION

It is necessary to offer an alternative to the state of the art which covers the gaps found therein, particularly related to the lack of proposals which really permit the attaching to a platform, via a gateway, of a device which is unknown for the gateway, i.e. the gateway does not have previous information about it.

To that end, the present invention concerns, in a first aspect, to a method for attaching a wireless or non-wireless device to a platform and establishing communication there with, comprising providing a gateway, which is able to communicate with said platform, with at least configuration information regarding a device to be attached thereto.

On contrary to known proposals, in a characteristic manner, in the method of the first aspect of the invention said device is unknown for said gateway, and the method further comprises:
providing a Dynamic Pair-up function in said gateway;
using, the gateway, said Dynamic Pair-up function to retrieve said configuration information from a data source; and
using, the gateway, said retrieved configuration information to automatically establish communication with said device.

For a preferred embodiment, said data source is said platform.

Depending on the embodiment, said descriptor file is a XML file, a JSON file, a plain text file with standard or proprietary formatting, a binary file with standard or proprietary formatting, or any other kind of file a person skilled in the art would consider appropriated for the indicated purpose.

Other embodiments of the method of the first aspect of the present invention are described according to appended claims 3 to 14, and in a subsequent section related to the detailed description of several embodiments.

A second aspect of the invention concerns to a system for attaching a wireless or non-wireless device to a platform and establishing communication there with, comprising:
a device;
a platform;
a gateway which is able to communicate with said platform; and
means for providing said gateway with at least configuration information regarding a device to be attached thereto.

On contrary to the known systems, in the one provided by the second aspect of the invention, in a characteristic manner, said device is unknown for said gateway, and:
said means for providing said gateway with at least configuration information comprises a Dynamic Pair-up function implemented in the gateway;
the gateway is configured to retrieve said configuration information from a data source using said Dynamic Pair-up function; and
the gateway is also configured for using said retrieved configuration information to automatically establish communication with said device.

For a preferred embodiment, said data source is said platform.

The platform is, depending on the embodiment, a remote server or any kind of data source the gateway is able to communicate with (USB stick, smart card, RF chip, etc.).

Other embodiments of the system of the second aspect of the present invention are described according to appended claims 17 to 22, and in a subsequent section related to the detailed description of several embodiments.

By means of the proposed invention adding a new device to a platform (i.e. regardless of the field, Tele-Health, Tele-Care, Smart-Homes, etc.) does not take more than 10 minutes. The basic performance would be an operator attaching remotely a new sensor into the system, and then, the patient only would have to use the device from the very first moment, avoiding the typical setting up problems. Thus, adding new sensors from different brands (i.e. AND, OMRON, Nonin etc.), technologies (i.e. USB, Bluetooth, ANT+, Zigbee, etc.) and data schemes is very straight forward now and this is the fruit of the innovation results coming out from implementation of the SARA system, a Tele-Health platform intended for dealing with chronic diseases which will be described in more detail in a posterior section, to which different kind of sensors can be connected to, such as a Bluetooth pressure sensor, a USB glucometer, a WiFi blood-pressure meter, a Zigbee accelerometer, a RS232 light sensor, etc.

The present invention differs from US20100016683 in that said patent application describes a method to set up a monitoring system whereas the Dynamic Pair-up function of the method and system of the present invention is focused on the acquisition of data that subsequently can be processed and use it in a platform of any kind. Moreover, as stated above, US20100016683 system knows beforehand which biometric devices can communicate with and how to perform the communication, while, in the present invention, the gateway, remarkably, does not know beforehand anything about the device, including its communication protocol. Because of that, the gateway of the present invention needs to be provided with the device description from a data source, including communication approach, information to establish connection with the device and the used protocol to exchange data. The system of US20100016683 does not need this additional information, only a hardware id, because they know their devices already.

In summary, the Dynamic Pair-up function describes a way to integrate new unknown devices and that is different from US20100016683, which is describing how to set up a monitoring system including already-integrated biometric devices and not focusing on the process of attaching the devices to the system.

Regarding EP1850527 system, as stated above, the main focus of system described therein is describing a way to provide and set up services that knows beforehand while the Dynamic Pair-up function of the method and system of the present invention describes the process of how to acquire data from unknown sources/sensors/devices from the very beginning when the new sensor/device is not still attached to the system. Furthermore, in the system of EP1850527 services must be enabled and provided with some configuration information in order for the gateway to communicate with them. On the contrary, the Dynamic Pair-up function installed in the gateway retrieves the configuration information itself from a data source and subsequently, it is able to establish communication with the new sensor automatically.

### BRIEF DESCRIPTION OF THE DRAWINGS

The previous and other advantages and features will be more fully understood from the following detailed description of embodiments, with reference to the attached drawings which must be considered in an illustrative and non-limiting manner, in which:

FIG. 1 shows a Tele-Health System according to an embodiment of the system of the second aspect of the invention;

FIG. 2 shows the Dynamic Pair-up Function State Machine of the method and system of the present invention;

FIG. 3 show different implementations of the Connector Component of the method and system of the invention, called SARA Connector Component for the implementation there illustrated;

FIG. 4 is an implementation of the SARA Connector component;

FIG. 5 shows the internal view of the Dynamic Pair-up function of the present invention; and

FIG. 6 shows a main performance flow of an embodiment of the method of the first aspect of the invention.

### DETAILED DESCRIPTION OF SEVERAL EMBODIMENTS

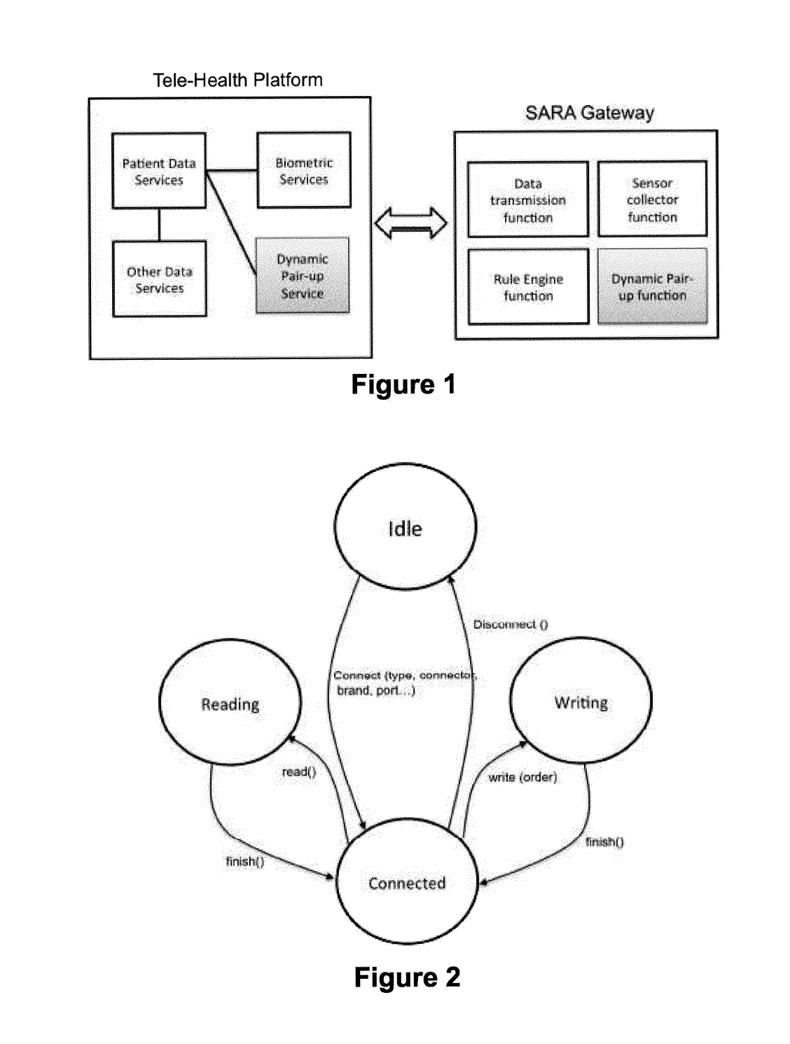

The Dynamic Pair-up function is integrated within the Tele-Health system that Telefonica has implemented according to an embodiment of the present invention. The fast attachment of new sensors and integration with the platform is paramount for this system and that gives it its uniqueness in the market. This Tele-Health system is made out of two different parts, the platform and the here called SARA Gateway. The platform collects all the information and provides tools to visualize data and the Gateway collects information from the sensors and forwards it to the Platform. The piece of innovation is mainly focused in the client side. FIG. 1 gives an overview of this system.

The real innovative part of this system, and of the method it implements, is provided for what is here called the “Dynamic Pair-up function”. This function gives a mean to add new sensors to the platform minimizing the development impact and so, speeding up new sensor integrations. This function is focused in the client side, i.e. the SARA Gateway, although there is some support in the server side to download some descriptor files (i.e. in FIG. 1—Dynamic Pair-up service). The Dynamic Pair-up function will be in charge of:
Adding new sensors with a remote, dynamic and a fast approach.
Integrating new communication technologies (i.e. USB, Bluetooth, Zigbee, Irda, etc.).
Integrating new sensor brands (i.e. Nonin, AND, Omrom, etc.).
Managing communication with the sensors.
Providing an understandable input to the SARA Gateway so that the sensors' measures can be stored in the platform.

In the server side, the Dynamic Pair-up service, provides remote services to provide the SARA Gateway with the descriptor files needed by the Dynamic Pair-up function. The Dynamic Pair-up service will be in charge of:
Hosting the descriptor files.
Allowing the addition of new descriptor files for brand new sensors.
Providing information about what sensors are going to be used on each SARA Gateway.
Providing the descriptor files to SARA Gateways on demand.

In order to simplify the addition of new sensors and the communication management between the SARA Gateway and the sensors; the Dynamic Pair-up Function defines a layer of abstraction with four different performance states. Basically, an state idle when there is no connection between the SARA Gateway and the sensor, a connected state when the connection has been established, a reading state when sensor data is collected by the SARA Gateway and write when there is an action from the SARA Gateway to the sensor. FIG. 2 shows this performance using a state machine diagram.

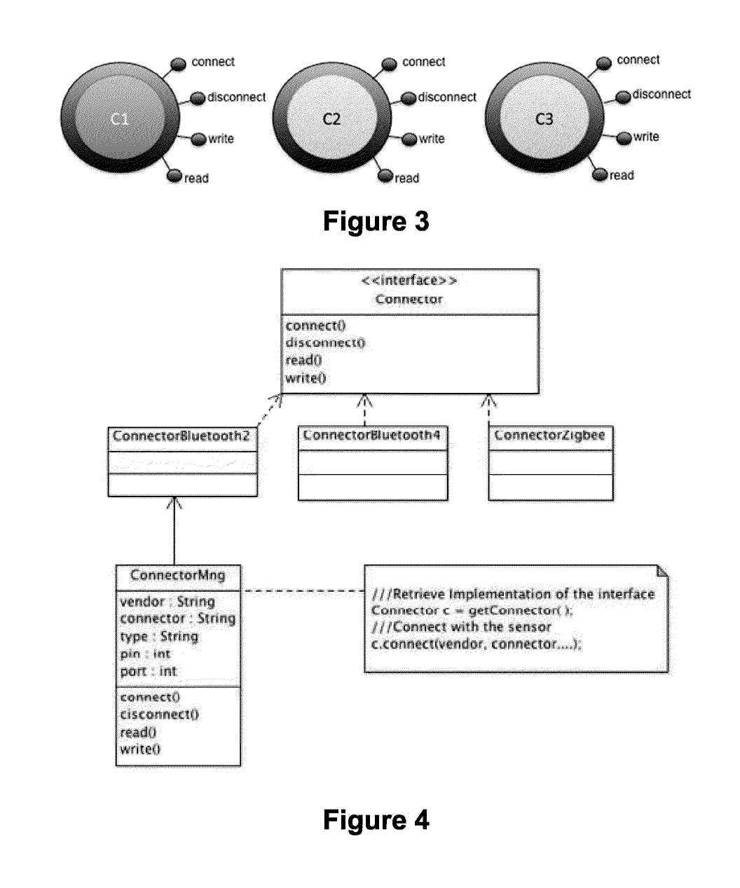

Transitions between the different states of the function are implemented using the SARA Connector component. This component is wrapped using a common interface so that only specific implementations are needed when the time comes to add a new communication technology. FIG. 3 shows three different components that share the same interface and have specific implementations to manage communications to lower level.

As shown in FIG. 3, the SARA Connector component provides the following interface:
Connect: that is the action to start communication SARA Gateway—sensor.
Disconnect: finish communication between SARA Gateway and sensor.
Read: SARA Gateway starts collecting data from the sensor.
Write: SARA Gateway sends a command to the sensor.

The implementation of the SARA connector component is done by following the classic interface pattern so that specific developments can be done keeping the save interface. Thus, whenever a new communication technology must be added, only the implementation of the defined methods (i.e. connect, disconnect, read and write) is necessary to manage the information with the source, that is to say, the sensor. FIG. 4 shows the class diagram of the SARA connector component.

Therefore, if there is already a connector supporting the communication technology of the new sensor we want to integrate in the platform, the only thing the Dynamic Pair-up function needs is to be handed over with the right parameters for that specific vendor (i.e. AND, Nonin, etc.) and that kind of device (i.e. weigh scale, blood pressure, pulsioximeter, etc.), and also some parameters related to the connection like the port, pin, if it is the case. As well as that, information related of how the incoming datagram is structured for this brand/type of device must be provided so that the parser is able to extract the information from the source and eventually forward the device measures (i.e. blood pressure, weigh, etc.) to the platform. Considering that communication technologies change at much lower rate (i.e. Bluetooth v2 has been around for more than 6 years now and still many devices support this technology) than sensor brands/type/data schemes, the Dynamic Pair-up function provides a very straight way to integrate new devices. In most of the cases, the only needed action is to provide a descriptor file with all the information mentioned before, so that the function is able to connect with the sensor, get the information, parse it and deliver it to the platform.

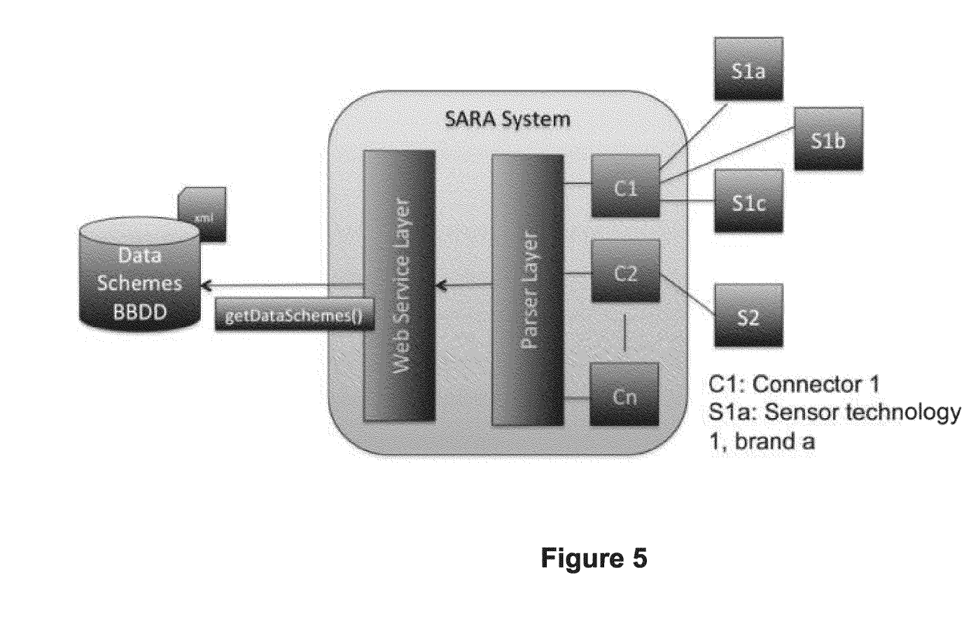

FIG. 5 shows an extended view of the Dynamic Pair-up function according to the last paragraph description.

FIG. 5 shows the three main parts that make out the Dynamic Pair-up function (i.e. in the SARA Gateway). As it was anticipated, the innovation is in the client side (i.e. SARA Gateway), although the server side deals with the important process of adding the device remotely, but this is not the focus of this patent:
Array of connectors: This layer is the responsible to communicate with the sensors
Parser: This module is able to extract the collected information by using the descriptor file. This latter is automatically downloaded from the platform by the Dynamic Pair-up function since it is associated to a specific patient's kit (i.e. devices around the patient).
Communication with the platform: This layer provides the communication capability with the platform, both to download the files describing the sensors' datagrams and connection and to send captured measures to the platform.

The last three main parts can also represent three respective hardware units of the system of the second aspect of the invention for an embodiment.

Therefore, whenever there is a new sensor, the technical operator of the platform will add a new file describing the device so that the SARA Gateway can download the description, connect with the sensor and understand what it says. In the Telehealth Platform the descriptor files are encoded using XML. The main tags of this XML are described in the next bullet points:
gtsensor: In this tag, all the different attributes related to the communication with the sensor are described.
Name: This is the name of the kind of device (e.g. pulsioximeter, weigh scale, etc.)
Vendor: The name of brand
Type: This attribute describes the connector's technology (e.g. Bluetooth, Zigbee, Irda, etc.).
Mode: This is for telling whether the gateway is acting as a client or a server
Timeout: Maximum waiting time for retrieving data from the sensor
MaxFrames: Maximum number of received measures before forcing disconnection
Port, mac, pin are attributes specific for the Bluetooth technology
parser: This tag helps the parser module to understand how it can read the info.
calib: This is an optional tag and it indicates to the Dynamic Pair-up function that the sensor needs calibration

Then, within parser and calib, tags like “value”, “condition”, “constraint”, etc., can set up incoming data. For writing data, the command “send” is used. The “sleep” tag is for forcing a waiting time. The description of these tags is out of the scope of the invention.

Next how a XML, describing datagram and connection, looks like:

```
<gtsensor name=“pulsioximeter” vendor=“nonin” type=“bluetooth” mode=“client” descriptorname=“ ”
port=“1” mac=“00:1c:05:00:45:e0” pin=“163300” timeout=“30000” maxframe=“1”>
    <parser>
        <tag type=“send” value=“68” once=“1” />
        <tag type=“send” value=“56” once=“1” />
        <tag type=“constraint” byteoffset=“1” datatype=“16” source=“7” value=“1” />
        <tag type=“value” name=“ ” byteoffset=“0” source=“6” datatype=“16” />
        <tag type=“value” name=“ ” byteoffset=“0” source=“5” datatype=“16” />
        <tag type=“constraint” byteoffset=“1” datatype=“16” source=“7” value=“0” />
        <!-- HEART RATE -->
        <tag type=“compoundvalue” byteoffset=“0” name=“HeartRate” measuretype=“6” datatype=“1”>
            <sub type=“value” source=“0” dest=“0” byteoffset=“−1” datatype=“16” />
            <sub type=“value” source=“1” dest=“1” byteoffset=“−1” datatype=“16” />
            <sub type=“value” source=“2” dest=“2” byteoffset=“−1” datatype=“16” />
            <sub type=“value” source=“3” dest=“3” byteoffset=“−1” datatype=“16” />
            <sub type=“value” source=“4” dest=“4” byteoffset=“−1” datatype=“16” />
            <sub type=“value” source=“5” dest=“5” byteoffset=“−1” datatype=“16” />
        </tag>
        <!-- OXIMETER -->
        <tag type=“value” name=“SP02” byteoffset=“1” datatype=“1” measuretype=“7” />
        <tag type=“constraint” byteoffset=“1” datatype=“16” source=“7” value=“0” />
        <!-- BATTERY -->
        <tag type=“compoundvalue” byteoffset=“0” name=“battery” measuretype=“21” datatype=“1”>
            <sub type=“value” source=“0” dest=“0” byteoffset=“−1” datatype=“16” />
                    <sub type=“constant” operation=“*” value=“−1” />
                <sub type=“constant” operation=“+” value=“1” />
            <sub type=“constant” operation=“*” value=“100” />
        </tag>
        <!-- END -->
        <tag type=“constraint” byteoffset=“0” datatype=“16” source=“7” value=“0” />
    </parser>
</gtsensor>
```

The XML depicted above corresponds to a Nonin Bluetooth pulsioximeter. As explained previously, the first line describes all the information related to the communication. The connector will use those parameters to get communication with the sensor. Then, as soon as the connection is ready and data starts to reach the SARA Gateway, the next important part of the XML is the one nested by the tag “parser” and those are used by the Dynamic Pair-up function to understand how to extract the information, parse it and provide the measures to the platform. Thus, whenever we want to add a new device to the platform, the only thing it is needed, as long as we have already the connector, is to provide this XML to the Dynamic Pair-up. That usually takes about 10 minutes of work which is a very remarkable achievement considering the effort that takes integrating new devices.

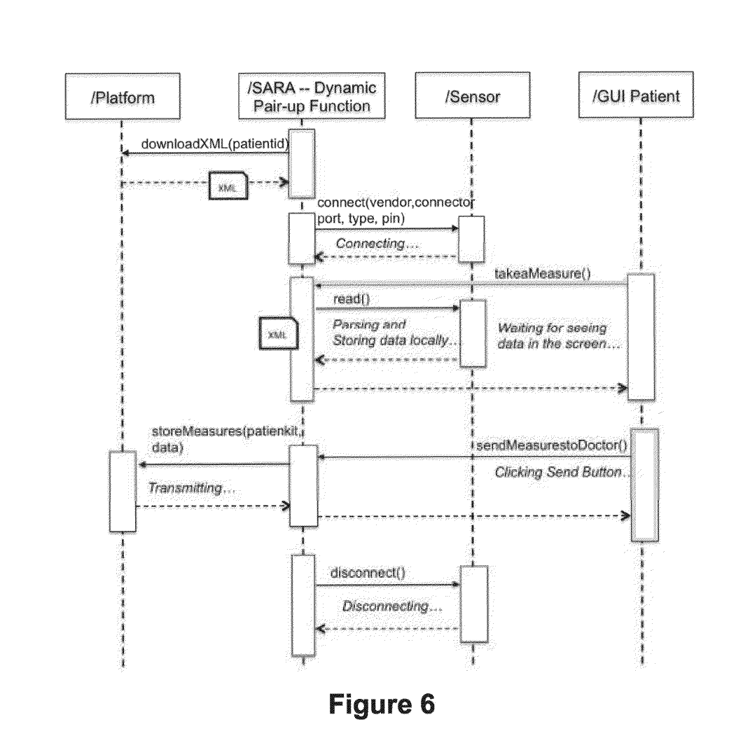

In order to finalize with the detailed explanation of the Dynamic Pair-up function, FIG. 6 shows the normal performing of the system when a new sensor is added.

The first thing the Dynamic Pair-up function does is to download the descriptor files associated to the patient in order to be able to connect with the sensor, and latter, to parse the collected data when the patient starts taking a measure. Once done that, the information is stored locally in the SARA Gateway. When the patient clicks the button “Send information to the doctor”, then the measures are sent to the platform for doctor's visualization. Finally, the Dynamic Pair-up function disconnects from the sensor.

### ADVANTAGES OF THE INVENTION

The invention has the following advantages:
Gives an alternative to Continua Alliance standards. Nowadays, they are only few Continua devices in the market and the ones implementing the standard are very expensive.
Speeding up integration of new sensors.
No need of implementation, as long as the connectors are there, but editing a descriptor file with the sensor's connection and datagram information and adding it remotely into the platform.
No need to release a new version of the client, which is a very tedious process of installation in each patients' PC, every time a new sensor is integrated
Improve Tele-Health platform flexibility to integrate new brands, technologies and data schemes.
Speeding up time to market.

A person skilled in the art could make changes and modifications to the here described embodiments without departing from the scope of the invention as defined in the appended claims.

### ACRONYMS AND ABBREVIATIONS

API Application Program Interface
USB Universal Serial Bus
IRDA Infrared Data
PC Personal Computer
XML Extensible Markup Language
Zigbee Zonal Intercommunication Global-standard, long Battery, Economical and Efficient

### REFERENCES

[1] Wearable devices market: http://lirnitlesstechnology.com/gadgets-monitor-health-fitness
[2] http://en.wikipedia.org/wiki/Bluetooth_low_energy

1. A method for attaching a wireless or non-wireless device to a platform and establishing communication there with, comprising providing a gateway, which is able to communicate with said platform, with at least configuration information regarding a device to be attached thereto, the method being characterised in that said device is unknown for said gateway, and in that the method further comprises:
providing a Dynamic Pair-up function in said gateway;
using, the gateway, said Dynamic Pair-up function to retrieve said configuration information from a data source; and
using, the gateway, said retrieved configuration information to automatically establish communication with said device.

2. The method of claim 1, wherein said data source is said platform.

3. The method of claim 1, wherein said configuration information includes at least communication approach, information to establish connection with the device and the used protocol to exchange data.

4. The method of claim 1, wherein said Dynamic Pair-up function defines a common interface layer of abstraction with four different performance states: an state idle when there is no connection between the gateway and the device, a connected state when the connection has been established, a reading state when device data is collected by the gateway and a writing state when there is an action from the gateway to the device.

5. The method of claim 4, wherein said common interface includes the next actions or commands to implement the transitions between said different performance states:
Connect: that is the action to start communication between gateway and device;
Disconnect: with this action finishes communication between gateway and device;
Read: with this action gateway starts collecting data from the device; and
Write: with this action gateway sends a command to the device.

6. The method of claim 5, comprising sending said connect command from the gateway to the device with the right parameters according to at least part of the retrieved configuration information, including Vendor and connection parameters.

7. The method of claim 5, comprising, the gateway, parsing the data collected from the wireless device by using the retrieved configuration information.

8. The method of claim 7, comprising sending the parsed data to said platform.

9. The method of claim 7, wherein said configuration information includes information related to how the incoming datagram, from the device, is structured for the brand/type of device so that said parsing is able to extract the information from the source and eventually forward the device measures to the platform.

10. The method of claim 1, comprising using, the gateway, said Dynamic Pair-up function to retrieve said configuration information by sending, to said platform, a download request for an object or area to which at least said device is associated.

11. The method of claim 1, wherein said wireless or non-wireless device is a sensor.

12. The method of claim 1, comprising using, the gateway, said Dynamic Pair-up function to retrieve said configuration information by sending, to said platform, a download request for an object or area to which at least said device is associated;
wherein said wireless or non-wireless device is a sensor; and
wherein it is applied to a Tele-Health system where said device is associated to a patient for monitoring any biologic parameter thereof, said object being said patient and said download request including an identification of the patient.

13. The method of claim 1, wherein said configuration information is provided by means of at least one descriptor file.

14. The method of claim 13, wherein said descriptor file is at least one of a XML file, a JSON file, a plain text file with standard or proprietary formatting and a binary file with standard or proprietary formatting.

15. A system for attaching a wireless or non-wireless device to a platform and establishing communication there with, comprising:
a device;
a platform;
a gateway which is able to communicate with said platform; and
means for providing said gateway with at least configuration information regarding a device to be attached thereto,
the system being characterised in that said device is unknown for said gateway, and in that:
said means for providing said gateway with at least configuration information comprises a Dynamic Pair-up function implemented in the gateway;
the gateway is configured to retrieve said configuration information from a data source using said Dynamic Pair-up function; and
the gateway is also configured for using said retrieved configuration information to automatically establish communication with said device.

16. The system of claim 15, wherein said data source is said platform.

17. The system of claim 16, where said platform is a remote server or any kind of data source the gateway is able to communicate with.

18. The system of claim 15, wherein the gateway comprises a plurality of Connectors components wrapped using a common interface operating or configurable to operate, via specific implementations of the common interface, through different communication technologies.

19. The system of claim 18, where said retrieved configuration information includes information for allowing the gateway to perform said specific implementations of the common interface.

20. The system of claim 18, wherein the gateway further comprises a parser unit connected to said plurality of connectors for collecting and parsing data coming therefrom parsing the information coming therefrom.

21. The system of claim 20, where the gateway further comprises a unit for communicating with the platform to download said configuration information and to send the parsed data.

22. The system of claim 15, where said gateway is configured for implementing the actions of the following method for attaching a wireless or non-wireless device to a platform and establishing communication therewith:
providing the gateway, which is able to communicate with said platform, with at least configuration information regarding the device to be attached thereto, said device is unknown for said gateway:
providing the Dynamic Pair-up function in said gateway;
using, the gateway, said Dynamic Pair-up function to retrieve said configuration information from a data source; and
using, the gateway, said retrieved configuration information to automatically establish communication with said device.
